(19) United States
(12) Patent Application Publication
(10) Pub. No.: US 2025/0246285 A1
(43) Pub. Date: Jul. 31, 2025

(54) APPARATUS FOR GLYCEMIC CONTROL

(71) Applicant: INSULET CORPORATION, Acton, MA (US)

(72) Inventors: Matthew ALLES, Mooresville, NC (US); Joon Bok LEE, Acton, MA (US); Yibin ZHENG, Hartland, WI (US); Jason O'CONNOR, Acton, MA (US)

(21) Appl. No.: 19/035,537

(22) Filed: Jan. 23, 2025

Related U.S. Application Data
(60) Provisional application No. 63/627,497, filed on Jan. 31, 2024.

(51) Int. Cl. G16H 20/17; A61M 5/172; G16H 10/60; G16H 40/67

(52) U.S. Cl. CPC G16H 20/17; A61M 5/1723; G16H 10/60; G16H 40/67; A61M 2205/505; A61M 2230/20; A61M 2230/201

(57) ABSTRACT

Techniques, systems and devices for glycemic control are presented. An apparatus may include a display device and a processor in electronic communication with the display device. The apparatus includes a memory communicatively connected to the processor. The memory includes instructions configuring the processor to generate a user interface through the display device and receive user input through the user interface. The processor is configured to implement an activity mode of a plurality of activity modes of a wearable medical device based on the user input. The activity mode is indicative of temporary conditions affecting blood glucose levels of the user. The processor is configured to receive biological data from a user through a biological sensor in communication with the processor and calculate an amount of medication to deliver to a user based on the biological data and the implemented activity mode.

### CROSS-REFERENCE TO RELATED APPLICATIONS

This application claims priority to and the benefit of U.S. Provisional Application No. 63/627,497, filed Jan. 31, 2024, the entirety of which is incorporated herein by reference.

### FIELD

The present subject matter generally relates to glycemic control and blood glucose levels. In particular, the present subject matter relates to an apparatus for optimizing glycemic control.

### BACKGROUND

AID systems typically utilize a user's current glucose measurements, past insulin delivery history, and potential meal ingestion announcements to control the user's glucose concentrations. However, these parameters often show significant delays in exhibiting the impact of a wide range of disturbances to the glucose concentrations, and these delays often result in sub-optimal glucose control under such disturbances. These disturbances may result from many potential sources with a wide range of varying impact to users, such as ingestion of alcohol or other factors, initiation of stressful activities (such as performances), or others.

### SUMMARY OF THE DISCLOSURE

In an aspect, an apparatus for glycemic control is presented. The apparatus includes a display device and a processor in electronic communication with the display device. The apparatus includes a memory communicatively connected to the processor. The memory includes instructions configuring the processor to generate a user interface through the display device and receive user input through the user interface. The processor is configured to implement an activity mode of a plurality of activity modes of a wearable injection device based on the user input. The activity mode is indicative of temporary conditions affecting blood glucose levels of the user. The processor is configured to receive biological data from a user through a biological sensor in communication with the processor and calculate an amount of medication to deliver to a user based on the biological data and the implemented activity mode.

In another aspect, a system for glycemic control is presented. The system includes a wearable medical device including a biological sensor configured to generate biological data. The wearable medical device includes a liquid reservoir that stores medication. The wearable medical device includes an injector configured to administer the medication from the liquid reservoir to a user. The system includes a computing device in electronic communication with the wearable medical device. The computing device is configured to receive the biological data from the biological sensor. The computing device is configured to generate a user interface through a display device in electronic communication with the computing device and receive user input from the user interface. The computing device is configured to receive a user input and then implement an activity mode of a plurality of activity modes of the wearable medical device based on the biological data and the user input. The activity mode is indicative of temporary conditions affecting blood glucose levels of the user. The computing device is configured to communicate the implemented activity mode, or instructions related to the implemented activity mode, with the wearable medical device. The wearable medical device is configured to administer the medication to the user based on the implemented activity mode or the instructions related to the implemented activity mode.

These and other aspects and features of non-limiting embodiments of the present disclosed subject matter will become apparent to those skilled in the art upon review of the following description of specific non-limiting embodiments of the disclosed subject matter in conjunction with the accompanying drawings.

### BRIEF DESCRIPTION OF THE DRAWINGS

FIG. 1 illustrates a block diagram of an exemplary embodiment of an apparatus for glycemic control;

FIG. 2 illustrates a flow diagram of a process of glycemic control;

FIGS. 3A-D illustrate exemplary embodiments of graphical user interfaces according to the present disclosure;

FIG. 4A depicts an example of a wearable sensing device usable with the exemplary embodiments of graphical user interfaces according to the present disclosure;

FIGS. 4B-D depict exemplary embodiments of graphical user interfaces according to the present disclosure;

FIGS. 5A-D illustrate another embodiment of graphical user interfaces according to the present disclosure;

FIG. 6 illustrates represent a graph showing glycemic variations in a user when drinking;

FIG. 7 provides a graph representing glycemic variations of a user based on implementation an activity mode as described herein;

FIG. 8 illustrates a block diagram of an exemplary wearable drug delivery system suitable for implementing the described subject matter; and

FIG. 9 illustrates an exemplary embodiment of a block diagram of a machine learning module that may be used throughout this disclosure.

### DETAILED DESCRIPTION

In the following description, for the purposes of explanation, numerous specific details are set forth in order to provide a thorough understanding of the present disclosed subject matter. It will be apparent, however, that the present disclosed subject matter may be practiced without these specific details. As used herein, the word “exemplary” or “illustrative” means “serving as an example, instance, or illustration.” Any implementation described herein as “exemplary” or “illustrative” is not necessarily to be construed as preferred or advantageous over other implementations. All of the implementations described below are exemplary implementations provided to enable persons skilled in the art to make or use the embodiments of the disclosure and are not intended to limit the scope of the disclosure, which is defined by the claims.

At a high level, aspects of the present disclosure relate to glycemic control. Aspects of the present disclosure can be used to change operational parameters of a wearable injection device through selection of an activity mode. The activity mode may be presented to a user through a user interface. The user interface may display glycemic data and statuses of activity modes. In some embodiments, aspects of the present disclosure can be used to automatically determine and select an activity mode of a wearable injection device for a user, such as through machine learning. Many other aspects of the present disclosure may be found throughout the description below.

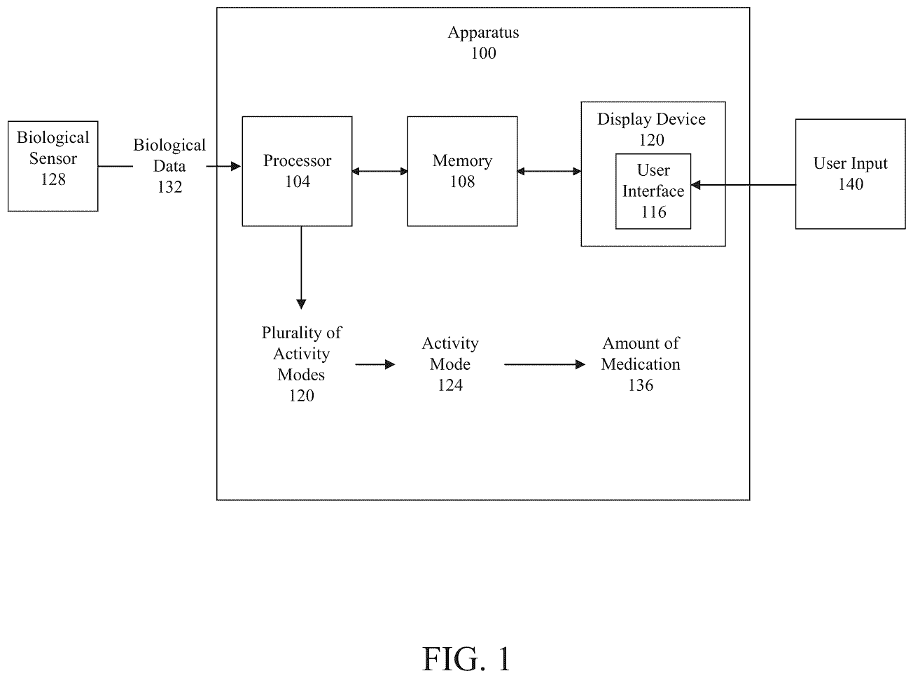

Referring now to FIG. 1A, apparatus 100 for glycemic control is presented. As used herein “glycemic control” means controlling blood glucose levels of a user. Apparatus 100 may include processor 104 and/or memory 108. As used in this disclosure, “communicatively connected” means connected by way of a connection, an attachment, or a linkage between two or more relata which allows for reception and/or transmittance of information therebetween.

Still referring to FIG. 1, in some embodiments, the apparatus 100 may be configured to receive biological data 132 from biological sensor 128. Apparatus 100 and/or processor 104 may be in electronic communication with biological sensor 128. “Electronic communication” as used in this disclosure is a form of connection between two objects where data is transferred. Electronic communication between biological sensor 128 and apparatus 100 may include, but is not limited to, wired, wireless, and/or other connections. A “biological sensor” as used in this disclosure is a device that detects biological data. Biological sensor 128 may include, without limitation, heart rate monitors, blood pressure sensors, blood oxygen sensors, thermometers, blood glucose monitors, continuous blood glucose monitors (CGM), ketone sensors, blood alcohol sensors and the like. Blood glucose monitor and continuous blood glucose monitors (CGM) may also be referred to as blood glucose meters. Biological sensor 128 may detect and/or generate biological data 132. “Biological data” as used in this disclosure is information pertaining to a user's biology. Biological data 132 may include, without limitation, temperatures, blood pressures, heart rates, hear rhythms, blood oxygen levels, blood glucose levels, and the like. Apparatus 100 may receive biological data 132 at processor 104. In some embodiments, apparatus 100 may receive biological data 132 from a Bluetooth, Wi-Fi, or other connection. Processor 104 may store biological data 132 in memory 108. Processor 104 may utilize biological data 132 that may be stored in memory 108 to determine one or more trends, patterns, and the like, as described in further detail below.

Apparatus 100 may include and/or be connected to display device 120. A “display device” as used in this disclosure is an object that displays information through a screen. The display device 120 of apparatus 100 may, in some embodiments, include a liquid crystal display (LCD), organic light emitting diode display (OLED), and/or other displays. The display device 120 may include a touchscreen. The touchscreen may be responsive to resistive touch, capacitive touch, and/or other forms of touch input. “Touch input” as used in this disclosure is a form of data communication through a touch sensitive device. Touch input may include, but is not limited to, user input such as tapping, double tapping, triple tapping, long presses, swipes, and the like. In some embodiments, touch input may include input received from one or more styluses. A “stylus” as used in this disclosure is an object configured to interact with a touchscreen. A stylus may include, but is not limited to, a capacitive stylus, resistive stylus, and the like. A user may interact with the touchscreen of the display 120 through a use of one or more styluses.

Apparatus 100 may display a graphical user interface 116 through display device 120. A “graphical user interface” as used in this disclosure is a form of communication with a computing device through one or more pictorial icons. The user interface 116 may include one or more event handlers. An “event handler” as used in this disclosure is a callback routine that operates asynchronously once an event takes place. An event handler of the user interface 116 may be linked with one or more graphical icons of a GUI of the user interface 116. For instance, and without limitation, an event handler may be programmed to generate a pop-up window of a settings menu upon a click of a settings icon displayed through the user interface 116. In some embodiments, the user interface 116 may include a mobile application, web portal, and/or other form of interface. The user interface 116 may display biological data 132, such as, without limitation, blood glucose levels, heart rate, ketone levels, blood oxygen, or the like.

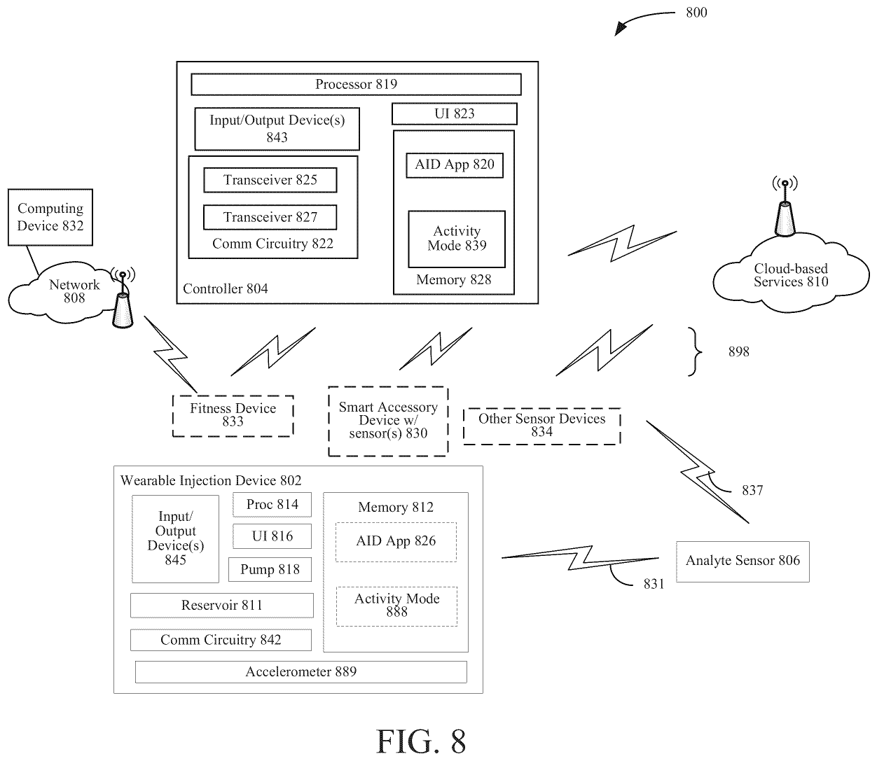

Still referring to FIG. 1, the user interface 116 may display or otherwise present one or more activity modes 124 of plurality of activity modes 120. An “activity mode” as used in this disclosure is an operational setting of a wearable medical device based on a user event(s) or activity(ies). A “wearable medical device” as used in this disclosure is a computing device affixed to a user that is configured to monitor and/or effect a user's health. Wearable medical devices may include, without limitation, smart watches, heart rate monitors, and the like. In some embodiments, a wearable medical device may include an insulin pump and/or other drug delivery system, such as, without limitation, the wearable drug delivery device described below with reference to FIG. 8. In some embodiments, the plurality of activity modes 120 may include one or more activity modes 124, such as, but not limited to, shopping modes, drinking modes, biking modes, running modes, travelling modes, feast modes, diet modes, competition modes, stress modes, and/or other modes. Each activity mode 124 of the plurality of activity modes 120 may correspond to different sets (operational setting) of operational parameters of a wearable medical device. “Operational parameters” as used in this disclosure are one or more settings of one or more processes of a wearable medical device. Operational parameters of the activity mode 124 may include one or more settings of a wearable drug delivery device, such as increasing or decreasing target blood glucose levels, increasing or decreasing maximum medication delivery, adjusting a total daily insulin (TDI) value, and the like. The activity mode may include a duration, such as, but not limited to, seconds, minutes, hours, and the like. A duration of the activity mode 124 may be calculated by the processor 104 and/or received from a user through the user input 140. For instance, the activity mode 124 may include a biking mode. The exercise of biking may reduce a user's blood glucose levels, which may make the user prone to hypoglycemia. The biking mode of the activity mode 124 may, for example, account for potentially lower blood glucose ranges of a user and decrease a TDI value for the user, reduce basal and/or bolus deliveries to the user, increase the target glucose of the system, reduce the maximum insulin delivery limits of the system, increase a frequency of readings of biological sensor 128, and/or adjust other operational parameters and set a biking duration, which may, for example, be 20, 30, 45 minutes or the like.

In some embodiments, processor 104 may categorize one or more activity modes 124 into “conservative,” “standard,” or “aggressive” operational parameters of a wearable medical device. In some embodiments, a user may categorize one or more activity modes 124 into one or more categories. For instance, and without limitation, a user may select one or more activity modes 124 and assign them to a category through user interface 116. A conservative operational parameter setting may include a blood glucose target value of about 120 mg/dL to about 150 mg/dL, a 25% decrease in a TDI value, a 25% decrease in basal and/or bolus dosage deliveries, and/or a 50% decrease of a maximum one-time delivery limit of a medication. A standard operational parameter setting may include an unchanged or default blood glucose target value, unchanged or default TDI value, and/or unchanged or default maximum one-time delivery limit of a medication. An aggressive operational parameter setting may include a blood glucose target set point of about 80 mg/dL to about 100 mg/dL, a 10% increase in a TDI value for a user, a 10% increase in basal and/or bolus dosage deliveries, and/or a 50% increase in a maximum one-time delivery limit. The following table shows the differences in exemplary parameter settings for each category:

| | | Algorithm | |
|---|---|---|---|
| | | Aggressiveness | Maximum |
| | Glucose target | parameter (such as | one-time |
| Parameter Setting | (mg/dL) | TDI) | Delivery limit |
| Conservative | 150 mg/dL | −25% | −50% |
| Standard | No change | 0% | 0% |
| Aggressive | 100 mg/dL | +10% | +50% |

In some embodiments, processor 104 may categorize the activity modes 124 to a conservative, standard, or aggressive parameter setting based on one or more types of activities. For instance, activities of the plurality of activity modes 120 may include one or more cardiovascular exercises. The processor 104 may determine cardiovascular activities of the plurality of activity modes 120 may correspond to aggressive parameter settings. The processor 104 may determine activities such as commuting to work, shopping, meditating, and/or other activities of the plurality of activities 120 may correspond to conservative operational parameter settings. The processor 104 may determine activities of the plurality of activities 120 such as reading, drawing, watching a movie, and the like, may correspond to standard operational parameter settings. The processor 104 may utilize these categorizations to present a user with “preset” activity modes 124 through user interface 116.

In some embodiments, the processor 104 may select the activity mode 124 of the plurality of activity modes 120 based on the user input 140. In some embodiments, the operational parameters of the activity mode 124 may be specific to a specific activity mode 124. The processor 104 may, for instance, determine specific ranges of blood glucose targets, TDI value modifications, and/or maximum one-time delivery limits for a bowling activity of the activity mode 124. The specific operational parameters of the bowling activity of the activity mode 124 may deviate from the above conservative, standard, and/or aggressive operational parameter settings and may be unique to the activity of bowling. As a non-limiting example, specific operational parameters of a bowling activity of the activity mode 124 may include a blood glucose target value of 110 mg/dL, an increase in a TDI value of a user by 4%, and no change in a maximum one-time delivery limit. Continuing this example, the processor 104 may determine different stages of a bowling game and compare the biological data 132 accordingly. For instance, at the start of a bowling game, a user may have normal blood glucose levels, which may be received at the processor 104. The processor 104 may be operable to expect a slow decrease in blood glucose values of a user as they bowl. The processor 104 may determine, through the biological sensor 128, when the user is standing, sitting, bowling, and the like. Each of these parameters may be accounted for by the processor 104 to adjust operational parameters of a wearable medical device while the user is bowling. In some embodiments, the processor 104 may generate and/or retrieve a preset bowling activity mode 124 for a user.

“User input” as used in this disclosure is a form of data received from a user at a computing device. The user input 140 may include one or more touch inputs, as described above. The user input 140 may, in some embodiments, include entry of one or more text-fields, mouse inputs, keyboard strokes, and/or other inputs, without limitation. In an embodiment, processor 104 may display the plurality of activity modes 120 through the user interface 116 to a user. The user may provide the user input 140 in a form of selecting an activity mode 124 of the plurality of activity modes 120 through the user interface 116. The processor 104 may communicate the selected activity mode 124 to an external computing device, wearable medical device, and/or other device. The wearable medical device and/or other device, upon receiving the selected activity mode 124, may modify one or more operational parameters based on the selected activity mode 124.

In FIG. 1, the processor 104 may automatically determine the activity mode 124 of the plurality of activity modes 120 based on the biological data 132 and/or other data such as, without limitation, time of day, dates, locational data, and the like. The processor 104 may retrieve locational data, dates, times, and the like from the memory 108 and/or from one or more external computing devices in communication with the processor 104. The processor 104 may compare the biological data 132 to one or more biological thresholds. A “biological threshold” as used in this disclosure is a value or range of values that if met triggers a process of a wearable medical device. Biological thresholds may be stored in the memory 108 from the processor 104 and/or retrieved from one or more external computing devices. Biological thresholds may include values such as, but not limited to, values of blood glucose, heart rates, heart rhythms, temperatures, movements, sounds, and the like. The biological sensor 128 may include a wearable sensor, such as a smartwatch or other device, that may be configured to detect movements, locations, and the like. The processor 104 may compare data, such as the biological data 132, to one or more biological thresholds that may be stored in the memory 108.

For instance, and without limitation, the processor 104 may select a “jogging” activity mode of the activity mode 124 based on the biological data 132 showing increased heart rate of 140 beats per minute (bpm), a detected increase in movement through GPS tracking, and the like. In some embodiments, the processor 104 may communicate with a GPS or other location tracking system to determine one or more locations of a user. The processor 104 may utilize locations of a user to adjust durations of the activity mode 124. Continuing the above example, the processor 104 may start the selected activity mode 124 of jogging at a first location and exit and/or prompt an exit of the selected activity mode 124 of jogging once the user reaches a specific location, which may be set by the user and/or determined by the processor 104. The processor 104 may automatically communicate or otherwise instruct a wearable medical device to implement the selected activity mode 124. In some embodiments, the processor 104 may present a predicted or otherwise automatic generation of a selected activity mode 124 through the user interface 116. A user may provide the user input 140 in a form of accepting or rejecting the proposed selected activity mode 124 through a GUI of the user interface 116. The processor 104 may utilize the user input 140 for future predictions which may allow the processor 104 to become more accurate in subsequent processing.

In some embodiments, the processor 104 may communicate with one or more external databases. One or more external databases may store user data corresponding to activity modes 124. The processor 104 may communicate with one or more external databases through a cloud-computing network. The processor 104 may calculate a population “average” of biological data 132 corresponding to activity modes 124 based on data communicated with one or more external databases. The processor 104 may extract, sort, classify, or otherwise process data from one or more external databases, which may allow the processor 104 to identify and/or determine one or more data trends, patterns, and the like. The processor 104 may utilize a classifier, language processing model, and/or other processes to calculate one or more data patterns and/or data trends. A classifier may include a machine-learning model, such as a mathematical model, neural net, or a program generated by a machine learning algorithm known as a “classification algorithm,” that sorts inputs into categories or bins of data, outputting the categories or bins of data and/or labels associated therewith. A classifier may be configured to output at least a datum that labels or otherwise identifies a set of data that are clustered together, found to be close under a distance metric. Classification may be performed using, without limitation, linear classifiers such as without limitation logistic regression and/or Naive Bayes classifiers, nearest neighbor classifiers such as k-nearest neighbors classifiers, support vector machines, least squares support vector machines, fisher's linear discriminant, quadratic classifiers, decision trees, boosted trees, random forest classifiers, learning vector quantization, and/or neural network-based classifiers. The processor 104 may use a classifier to classify data communicated with one or more external databases into categories or groups. For instance and without limitation, the processor 104 may classify biological data to one or more activity modes, such as jogging, drinking, driving, and the like. Classification of the biological data 132 to one or more activity modes may allow the processor 104 to generate more accurate prompts of user interface 116 for a user to enter an activity mode 124. In some embodiments, classification of the biological data 132 may allow the processor 104 to determine averages of biological data 132 for a given activity mode 124. Averages of biological data 132 for a given activity mode 124 may allow the processor 104 to compare biological data 132 of a user to one or more standard deviations and report and/or display this comparison through the user interface 116 to the user.

A language processing model may be configured to extract, from one or more documents, one or more words. One or more words may include, without limitation, strings of one or characters, including without limitation any sequence or sequences of letters, numbers, punctuation, diacritic marks, engineering symbols, geometric dimensioning and tolerancing (GD&T) symbols, chemical symbols and formulas, spaces, whitespace, and other symbols, including any symbols usable as textual data as described above. Textual data may be parsed into tokens, which may include a simple word (sequence of letters separated by whitespace) or more generally a sequence of characters as described previously. A token refers to any smaller, individual groupings of text from a larger source of text. Tokens may be broken up by word, pair of words, sentence, or other delimitation. Tokens may in turn be parsed in various ways. Textual data may be parsed into words or sequences of words, which may be considered words as well. Textual data may be parsed into “n-grams”, where all sequences of n consecutive characters are considered. Any or all possible sequences of tokens or words may be stored as “chains”, for example for use as a Markov chain or Hidden Markov Model. A language processing model may produce associations between one or more words extracted from at least a document and detect associations, including without limitation mathematical associations, between such words, and/or associations of extracted words with categories of data, relationships of such categories to compatible label, and/or categories of compatible labels. Associations between language elements, where language elements include for purposes herein extracted words, categories of data, relationships of such categories to compatible labels, and/or categories of compatible label may include, without limitation, mathematical associations, including without limitation statistical correlations between any language element and any other language element and/or language elements. Statistical correlations and/or mathematical associations may include probabilistic formulas or relationships indicating, for instance, a likelihood that a given extracted word indicates a given category of data, a given relationship of such categories to compatible labels, and/or a given category of compatible label. For instance and without limitation, the processor 104 may use a language processing model to determine associations between words received through user input of the user interface 116 and one or more activity modes 124. As a non-limiting example, a user may input words through user interface 116 such as “long distance running”, “medium impact cardio”, “cross-country”, and the like which a language processing model may associate with an activity mode 124 of jogging. The processor 104 may utilize a language processing model to determine associations between words and activity modes of one or more populations of users.

The processor 104 may utilize a classifier and/or language processing model to generate and/or determine one or more data trends/patterns. Data trends and/or data patterns may include, but is not limited to, blood glucose values, target blood glucose values, TDI values, activity durations, time of activity engagement, medication delivery values, and the like. Further, classifiers, language processing models, or other processes may include the identification of different modes with similar impact to blood glucose, such as the use of large language models (LLM) to categorize multiple user entries that represent exercise as part of the same category for analysis-such as an activity mode titled “mountain biking” versus an activity mode titled “spin class.” For instance, the processor 104 may calculate (or determine) that a population average selects a shopping activity mode 124 of the plurality of activity modes 120 on weekends between 10 AM to 2 PM EST. In some embodiments, the shopping activity mode 124 may include a slightly aggressive increase in operational parameter settings of a wearable medical device, such as blood glucose targets, to account for long periods of walking and/or standing around, such as during a trip to a mall, which may lower blood glucose levels of a user. The processor 104 may compare the biological data 132 of a user to this trend and present a shopping activity mode 124 to a user through the user interface 116 during the above time window.

In some embodiments, the processor 104 may modify, over time, operational parameter settings of the activity mode 124 based on the user input 140 and one or more average population responses determined from the data stored in one or more external databases (not shown). Continuing the above example, the processor 104 may determine, based on data stored in one or more databases, that a user may shop from 10 AM to 11:30 AM on Saturdays. For instance, the processor 104 may receive biological data 132 that shows an increased heart rate. The processor 104 may communicate data through a personal management device (PDM), such as, without limitation, a smartphone of a user. The PDM may communicate data such as, without limitation, location, accelerometer data, and the like, which the processor 104 may use to determine one or more activities of the user. The processor 104 may correlate accelerometer data that may resemble a walking pattern of a user with an increased heart rate and a location of a mall to determine a user may be shopping, without limitation, and present a shopping activity mode 124 to the user through the user interface 116.

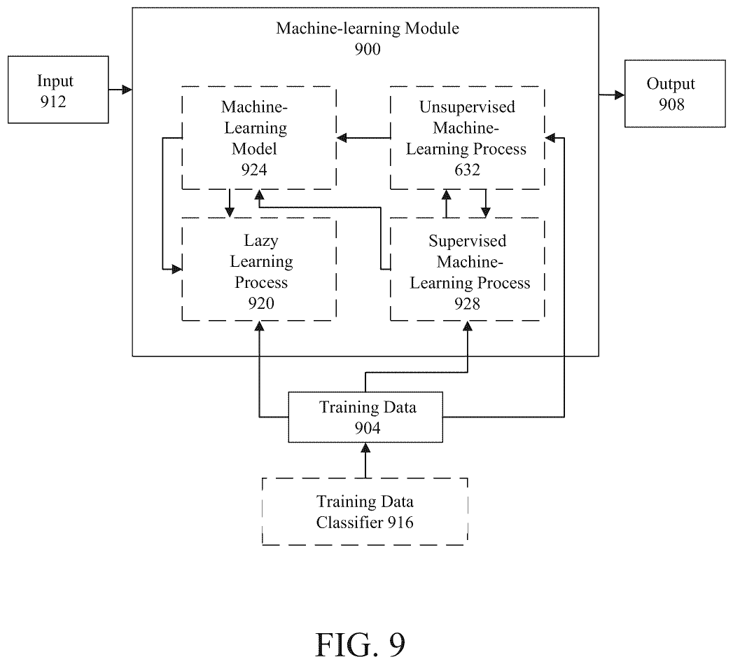

In a further example, the processor 104 may utilize an activity mode machine learning model to select the activity mode 124. An “activity mode machine learning model” as used in this disclosure is a machine learning process that outputs activity modes of a wearable medical device. The processor 104 may train the activity mode machine learning model with training data correlating biological data and user input to activity modes. Training data may be received through user input, external computing devices, and/or previous iterations of processing. The activity mode machine learning model may input the biological data 132 and/or the user input 140 and output a selected activity mode 124. The processor 104 may present a selected activity mode 124 to a user through user interface 116 based on the activity mode machine learning model. The activity mode machine learning model may further determine operational parameters of the selected activity mode 124. For instance and without limitation, the activity mode machine learning model may determine both that the selected activity mode 124 should be a basketball activity mode and that the selected activity mode 124 should increase a TDI value for the user by 40%. The activity mode machine learning model may be based on any machine learning model to provide the outputs to select an activity mode as discussed throughout this disclosure, such as below with reference to FIG. 9.

In an embodiment, the processor 104 may calculate one or more operational parameter settings specific to an activity of the activity mode 124. For instance, the activity mode 124 may include a “happy hour mode” or a drink consumption mode, such as alcohol or coffee. A “happy hour mode” as used in this example is a set of operational parameters of a wearable medical device configured for the consumption of alcohol. A “drink consumption mode” may similarly be used to modify a set of operational parameters of a wearable medical device. The processor 104 may suggest and/or select a happy hour mode of the activity mode 124 based on the biological data 132, the user's current location, and/or the user input 140. For instance, a user may provide the user input 140 that may include a selection of the activity mode 124 of a happy hour activity mode. In some embodiments, processor 104 may determine a selection of a happy hour mode of the activity mode 124 is appropriate based on the user's current location, as determined by the user's device, or the biological data 132. The biological sensor 128 may include, for example, a blood alcohol content (BAC) sensor. The BAC sensor may be affixed to a user and be operable to detect blood alcohol levels of the user and communicate them to the processor 104. The processor 104 may compare BAC values of a user to a BAC threshold which may initiate a happy hour mode of the activity mode 124. For instance, BAC threshold may include, but is not limited to, about 0.01% to about 0.2%, or The processor 104 may determine BAC values of a user exceed a BAC threshold and may automatically initiate a wearable medical device in an activity mode 124 of a happy hour mode.

Referring still to FIG. 1, the user input 140 may include a quantity of drinks, such as glasses or bottles of beers, shots, cups of coffee, and the like. The user input 140 may include a duration of drinking, such as, but not limited to, minutes, hours, and the like. The processor 104 may set a happy hour of the activity mode 124 based on the duration received from the user input 140. In some embodiments, the processor 104 may continually compare BAC levels of a user from the biological data 132 to extend or shorten a duration of a happy hour mode of the activity mode 124. The user input 140 may include a hardness factor of drinking. A “hardness factor” as used in this disclosure is a metric relative to a strongness (alcohol content) of an alcoholic beverage. A hardness factor may be on a scale from 1-5, 1-10, and the like, without limitation. For instance, on a scale from 1-5, 1 may indicate a “softest” drink and 5 may indicate a “hardest” drink. Softer drinks may include beverages such as, but not limited to, beers, margaritas, cocktails, and the like. Harder drinks may include beverages such as, but not limited to, gin, rum, vodka, whiskey, and the like. The processor 104 may prompt a duration, hardness, and/or other selection through user interface 116. A happy hour mode of the activity mode 124 may include operational parameter adjustments of a TDI value, such as, but not limited to, between about-10% to about 10%. The processor 104 may utilize a drinking machine learning model to determine operational parameter adjustments. A drinking machine learning model may include a machine learning model configured to input biological data and output operational parameters of a wearable medical device. A drinking machine learning model may be trained with training data correlating biological data to operational parameters. Training data may be received through user input, external computing devices, and/or previous iterations of processing. The processor 104 may input the biological data 132 to the drinking machine learning model which may output one or more operational parameters such as, but not limited to, TDI values, durations, blood glucose targets, maximum one-time delivery limits, and the like.

In FIG. 1, the processor 104 may calculate an amount of medicament or medication 136. An amount of medication or medicament as used in this disclosure is a quantity of medicine or drug. Exemplary medicaments or medication 136 that may be used include insulin, glucagon-like peptide-1 receptor agonist (GLP-1), glucose-dependent insulinotropic polypeptide (GIP), or other hormones, and/or combinations of medicaments, such as two or more of insulin, GLP-1, and GIP, or other like hormones. The amount of medication 136 may include a bolus of insulin or other medication. A bolus may include an upfront or immediate delivery of a medication amount. In some embodiments, the amount of medication 136 may include a duration of medication to be delivered to a user. A duration may include, without limitation, seconds, minutes, hours, and the like. For instance, and without limitation, the amount of medication 136 may include an initial delivery of 5 units of insulin, followed by a delivery of 1 unit of insulin every 10 minutes. The amount of medication 136 may include a total daily insulin (TDI) value, which may include a maximum amount of insulin a user may be administered. The processor 104 may adjust a TDI value of the amount of medication 136 based on the biological data 132, the selected activity mode 124, and/or other factors. The processor 104 may calculate the amount of medication 136 based on the selected activity mode 124 and/or the biological data 132. For instance, the biological data 132 may show that a user may have dropping blood glucose levels and the selected activity mode 124 may include a happy hour mode. The processor 104 may determine, based on the happy hour mode and the dropping blood glucose levels, to increase the amount of medication 136. The processor 104 may communicate the amount of medication 136 with an external computing device, such as, but not limited to, a laptop, smartphone, server, and/or other device. In some embodiments, the processor 104 may communicate the amount of medication 136 with a wearable medical device, such as the wearable injection device of FIG. 8 described below. A wearable injection device may include a liquid reservoir, a drug reservoir, a biological sensor, a pump, and a needle and/or cannula injector, without limitation. An injector may include, without limitation, a needle, cannula, syringe, and/or other piercing element in mechanical connection with a spring, pump, and/or other moving element. The apparatus 100 may communicate the amount of medication 136 to a wearable injection device, to which the wearable injection device may administer the amount of medication 136 to a user through an injector. In some embodiments, the apparatus 100 and/or the processor 104 may communicate instructions related to the selected activity mode 124 to a wearable medical device. Instructions may include administering of medication, raising or lowering of blood glucose threshold or target values, raising or lowering of insulin-on-board threshold or target values, and/or any other operation related to the selected activity mode 124 as described throughout this disclosure.

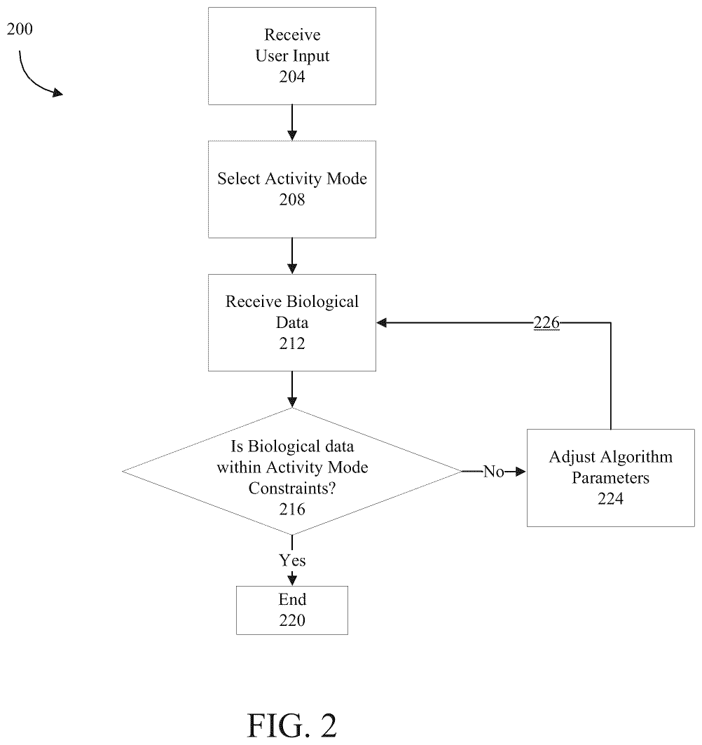

FIG. 2 illustrates an exemplary embodiment of a flowchart of an AID algorithm 200. At step 204 user input is received. A computing device may present one or more activity modes for a user to select through a user interface. The user input may include a selection of one or more activity modes through a user interface, such as described above with reference to FIG. 1. In some embodiments, the user input may include an intensity factor. An “intensity factor” as used in this disclosure is a metric pertaining to a strength of an activity. An intensity factor may include a value on a range of, but not limited to, 1-10, with 10 being the highest intensity. The user input may include a duration of an activity mode, such as, but not limited to, minutes, hours, and the like.

At step 208, the process selects an activity mode. The activity mode is selected based on the user input, as described above with reference to FIG. 1. The activity mode may include one or more constraints. Activity mode constraints may include one or more values of biological data, operational parameters, and the like. In some embodiments, process 200 may utilize Equation 1:

$$P = max ( min ( P \cdot \frac{{U}_{max} - U ( t )}{{U}_{max} - {U}_{min}} , {P}_{max} ) , {P}_{min} )$$

where P represents an AID parameter, Pₘₐₓ represents a maximum value, Pₘᵢₙ represents a minimum value. Uₘₐₓ represents a maximum user input intensity value, Uₘᵢₙ represents a minimum user input intensity value, and U(t) represents the user's input of the intensity of the activity. Process 200 may utilize Equation 1 or a similar weighting calculation to scale an increase or decrease in one or more parameters of an AID algorithm based on the user's selected intensity. For instance, P may represent blood glucose set points of an individual, insulin-to-carb ratio, and the like.

In some embodiments, process 200 may determine an acuteness parameter (also referred to as acuteness factor (AC)). An “acuteness parameter” as used in this disclosure is a metric pertaining to a sharpness of an event. An acuteness parameter may include a value of between about 0% to about 200%, without limitation. An acuteness parameter may indicate a relative impact to a user's body, with 0% being unchanged and 200% being a large impact. The acuteness parameter may be a metric relative magnitude of an activities (expected) impact on a user's blood glucose levels. For example, walking may have a lower “acuteness parameter” than running, as walking is expected to reduce the blood glucose level of a user, however, not to the extent that the same time of running would. The following table provides some examples of acuteness factors:

| Activity | Acuteness Factor (Ac) |
|---|---|
| Aerobic exercise (standard) | 100% |
| Strength training | 125% |
| Beer Drinking | 150% |
| Sickness | 25% |
| Performance | 175% |

Process 200 may determine and/or calculate one or more acuteness factors based on the user input, biological data, activity mode selected, and the like. The user's duration and intensity selections may be scaled by the acuteness factor. Equation 2 provides an exemplary formula for scaling an intensity with an acuteness factor:

$${U}_{final} ( t ) = ( 1 + Ac ) \cdot U ( t )$$

where U_{final} represents a final intensity value and Ac represents an acuteness factor. Process 200 may use Equation 2 to modify an intensity factor received from a user based on an acuteness of the activity. For instance, an acuteness of a bike ride may include a value of 100%, which may increase an intensity of an activity mode.

Equation 3 provides an exemplary formula for scaling a duration with an acuteness factor:

$${D}_{final} ( t ) = \frac{1}{( 1 + Ac )} \cdot D ( t )$$

where D_{final} represents a final duration value, Ac represents an acuteness factor, and D(t) represents a duration input from the user. Process 200 may use Equation 3 to scale a duration of an activity mode based on an acuteness of an activity. For instance, a sick mode may include an acuteness factor of 25%, which may increase a total duration of a sick mode. In other words, a larger duration would represent a lower acuteness and a larger acuteness would decrease a duration of an activity mode.

At step 212, biological data is received. Biological data may include blood glucose levels, temperatures, blood oxygen levels, blood alcohol content (BAC) levels, ketone levels, heart rates, heart rhythms, and the like, as described above with reference to FIG. 1. Biological data may be received from a biological sensor, such as a blood glucose monitor, heart rate sensor, and the like, without limitation.

At step 216, a comparison between the biological data with the activity mode constraints is made. A computing device may compare the biological data and/or operational parameters of a wearable medical device with the activity mode constraints.

At step 220, algorithm parameters are adjusted if the biological data is not within the activity mode constraints. For instance, if a blood glucose level is too high, one or more micro doses of medication may be delivered.

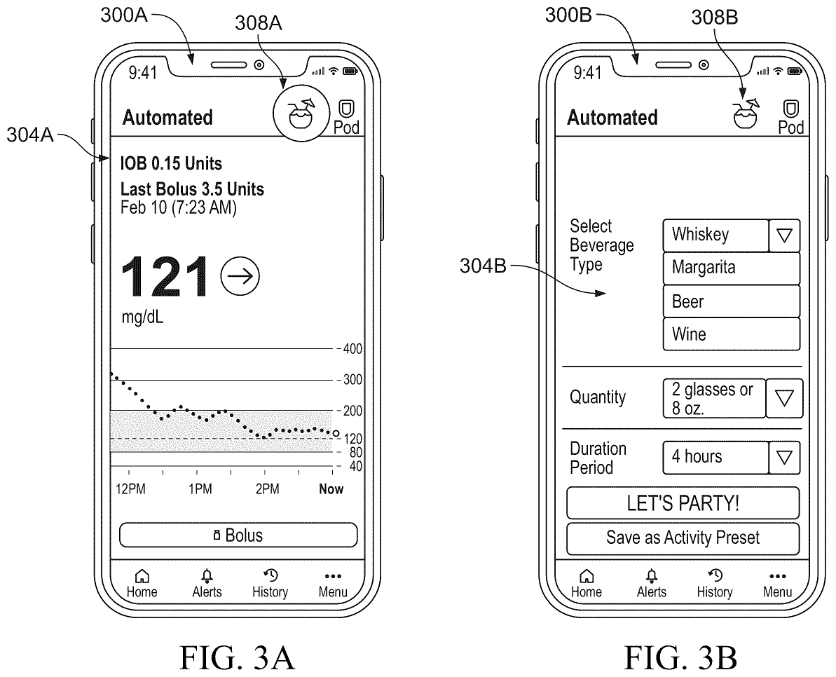

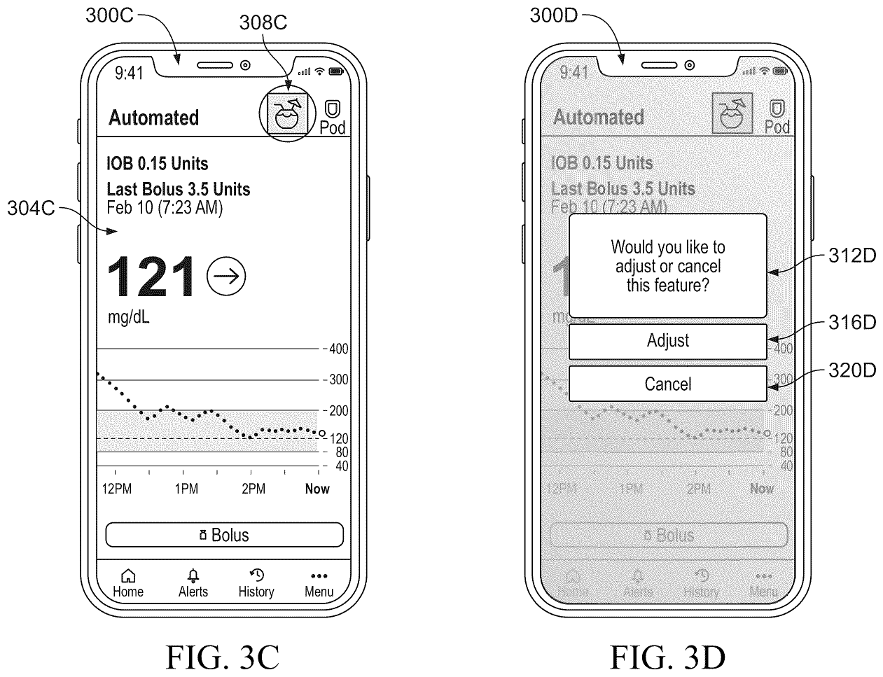

FIG. 3A represents a graphical user interface (GUI) 300A of an embodiment of the present disclosure. The GUI 300A may include glycemic data 304A. The glycemic data 304A may include, but is not limited to, blood glucose levels, medication delivery data, and the like. For instance, the glycemic data 304A may show a blood glucose value of 121 mg/dL and a trend arrow on a right side of the blood glucose value. The trend arrow may depict an increase, steadiness, or decrease in blood glucose values. The trend arrow may point to a right direction to depict a steadiness in blood glucose level, an upwards or diagonally upwards direction to indicate an increase in blood glucose levels, a downwards or diagonally downwards direction to indicate a decreasing blood glucose value, and the like. The glycemic data 304A may include a graph that may have an x-axis representing time and a y-axis representing blood glucose values. The GUI 300A may include activity indicator 308A. The activity indicator 308A may include a pictorial icon representative of an activity mode, for instance, an alcoholic beverage that may be representative of a happy hour mode, a coffee mug may be representative of a coffee drinking or drink consumption mode, a bicycle may be representative of a cycling mode, etc.

FIG. 3B depicts another GUI 300B of setting a happy hour mode. The GUI 300B may include user input field 304B. The user input field 304B may include one or more text-fields, drop down menus, and the like. A user may input a type of beverage, quantity of beverages, and/or a duration of a happy hour mode. The user input field 304B may include an activity mode activation button that may have a high contrast to a background of GUI 300B. An activity mode activation button may include text, such as, “Let's Party!”. The user input field 304B may include a save icon that may have a low contrast to a background of GUI 300B. A save icon may include a text box that may display text saying “Save as Activity Preset”. A user may engage with any of the above elements of the GUI 300B, without limitation. The GUI 300B may include activity indicator 308B, which may be similar to that of activity indicator 308A.

FIG. 3C represents a GUI 300C of an activation of a happy hour mode. The GUI 300C may display glycemic data 304C. The glycemic data 304C may be similar to that of glycemic data 304A. The GUI 300C may have activity indicator 308C. The activity indicator 308C may have a colored background, such as in a shape of a square. A colored background may include a color such as, but not limited to, red, blue, green, yellow, and/or any combination thereof. A colored background of the activity indicator 308C may indicate an activation of an activity mode. For instance, a green square of the activity indicator 308C may indicate an activity mode is active. A yellow square of the activity indicator 308C may indicate a wearable medical device is in a process of setting up an activity mode. A red square of the activity indicator 308C may indicate the activity mode is unavailable, the user is experiencing hypoglycemia, hyperglycemia, and the like.

Referring now to FIG. 3D, GUI 300D of a happy hour mode is presented. GUI 300D may include window 312D. The window 316 may include a pop-up or other window. The window 312D may include one or more sub-windows. For instance, the window 312D may include two sub-windows, 316D and 320D, positioned directly under the window 312D, where the sub-windows 316D and 320D represent user input. For instance, the GUI 300D may include a sub-window 316D, which may have an orange color and reads “Adjust” and a second sub-window 320D that may have a red color and reads “Cancel”. The window 312D may be positioned on top of GUI 300D, where GUI 300D may appear in a background with a lower brightness, contrast, and the like.

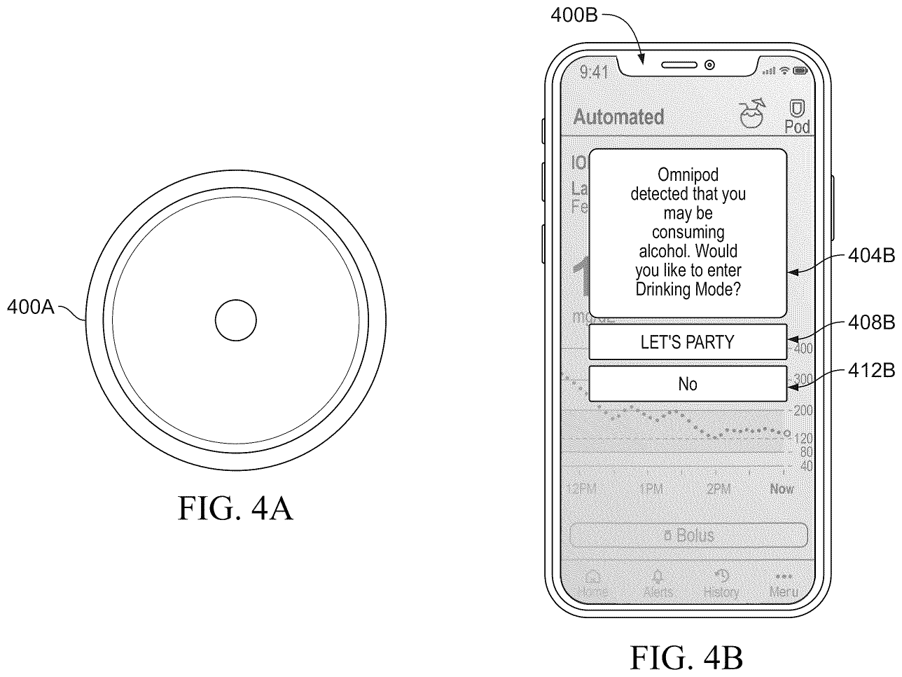

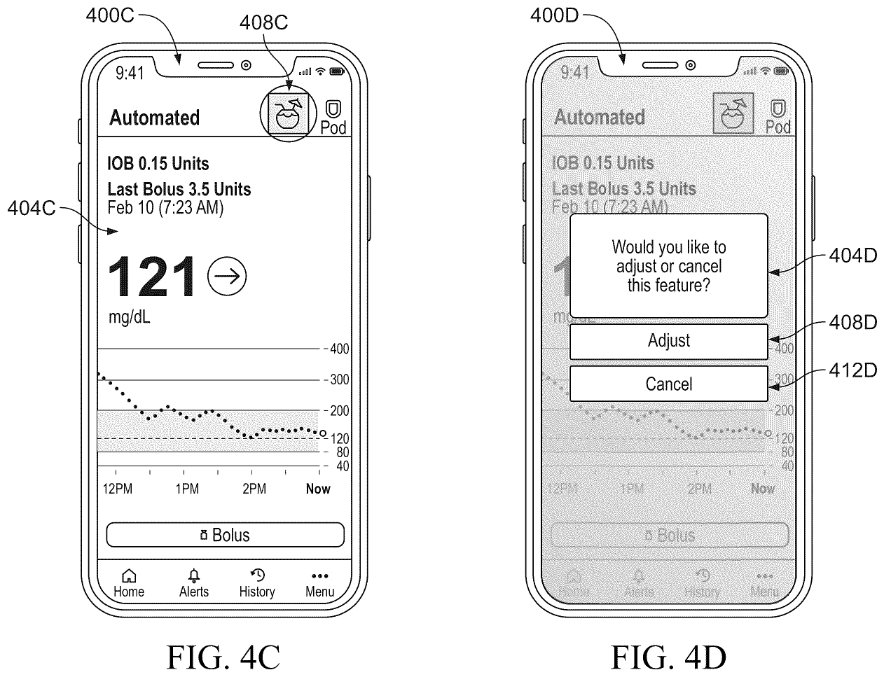

Referring now to FIG. 4A, an exemplary blood alcohol concentration (BAC) sensor 400A is presented. The BAC sensor 400A may include a wearable BAC sensor. For instance, the BAC sensor 400A may include an adhesive patch, wrist straps, and/or other affixing devices. The BAC sensor 400A may be configured to detect a blood alcohol level of a user. The BAC sensor 400A may detect a type of alcohol a user might be consuming, such as hard alcohol, beers, wine, and the like. The BAC sensor 400A may detect a type of alcohol based on increase and/or decrease in blood alcohol concentration of a user over a period of time. The BAC sensor 400A may detect levels of ethanol leaving a user's body and communicate this data to one or more computing devices, such as a smartphone, wearable medical device, and the like.

Referring to FIG. 4B, GUI 400B is shown illustrating an automatic happy hour mode. The GUI 400B may be configured to display window 404B. The window 404B may include a text box. For instance, the window 404B may include a text box alerting a user to a detected activity mode, such as a drinking mode. A prompt for a drinking mode may be generated by a computing device based on sensor data from a BAC sensor, such as the BAC sensor 400. The window 404B may display a prompt asking a user if they want to enter into a drinking mode, such as a happy hour mode. The window 404B may include two or more subwindows 408B and 412B. The first subwindow 408B may be positioned directly beneath the window 404B. The first subwindow 408B may represent a continue button. For instance, the first subwindow 408B may include a text box displaying “Let's Party” with an orange background. The GUI 400B may include second subwindow 412B. The second subwindow 412B may represent a cancel button. The second subwindow 412B may be positioned beneath the first subwindow 408B. The second subwindow 412B may include a text box displaying “No” with a red background.

Referring now to FIG. 4C, GUI 400C showing a happy hour mode is presented. GUI 400C may include and/or be similar to that of GUI 300A-D as described above with reference to FIGS. 3A-D. GUI 400C may display glycemic data 404C. The glycemic data 404C may be similar to that of the glycemic data 304A as described above with reference to FIG. 3A. GUI 400C may include activity indicator 408C. The activity indicator 408C may be similar to that of the activity indicator 308B as described above with reference to FIG. 3B.

Referring now to FIG. 4D, GUI 400D showing a user input field is presented. The GUI 400D may be similar to that of the GUI 300D as described above with reference to FIG. 3D. GUI 400D may include window 404D. The window 404D may be similar to that of window 404B as described above. The window 404D may include a pop-up or other window. The window 404D may include one or more text boxes. The window 404D may include a text box asking a user “Would you like to adjust or cancel this feature?”. GUI 400D may include first subwindow 408D and second subwindow 412D, each of which may be similar to the subwindows described above in FIG. 4B. The first subwindow 408D may include a text field reading “Adjust” with an orange background. The second subwindow 412D may include text reading “Cancel” with a red background. A user may interact with the first subwindow 408D, such as by tapping, clicking, or otherwise interacting with the first subwindow 408D. Interaction with the first subwindow 408D may animate GUI 400D to display a settings menu that may allow a user to adjust parameters of a happy hour. An interaction with the second subwindow 412D may include clicking, tapping, or other interactions. A user may interact with the second subwindow 412D, which may cause the window 404D, the first subwindow 408D, and/or the second subwindow 412D to disappear from GUI 400D.

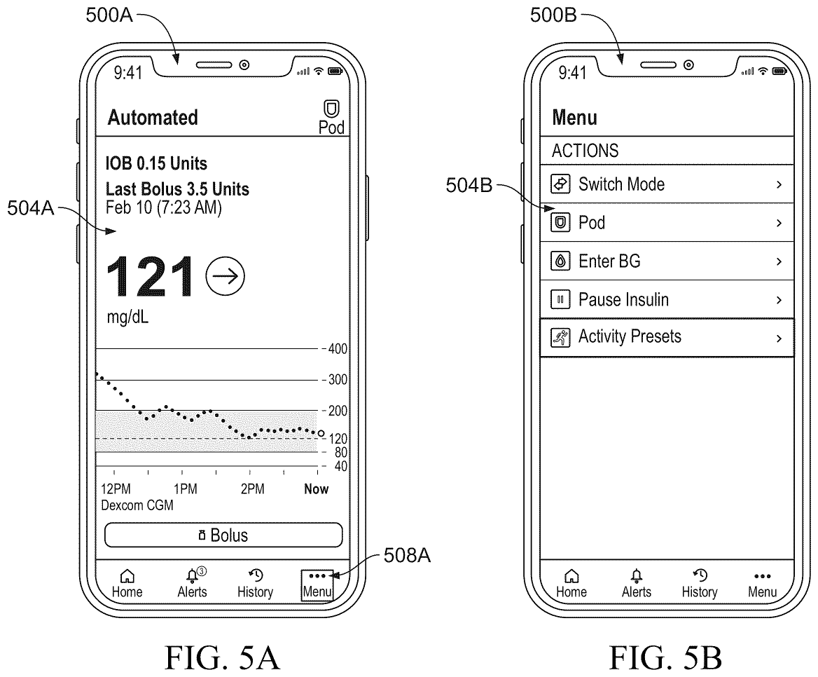

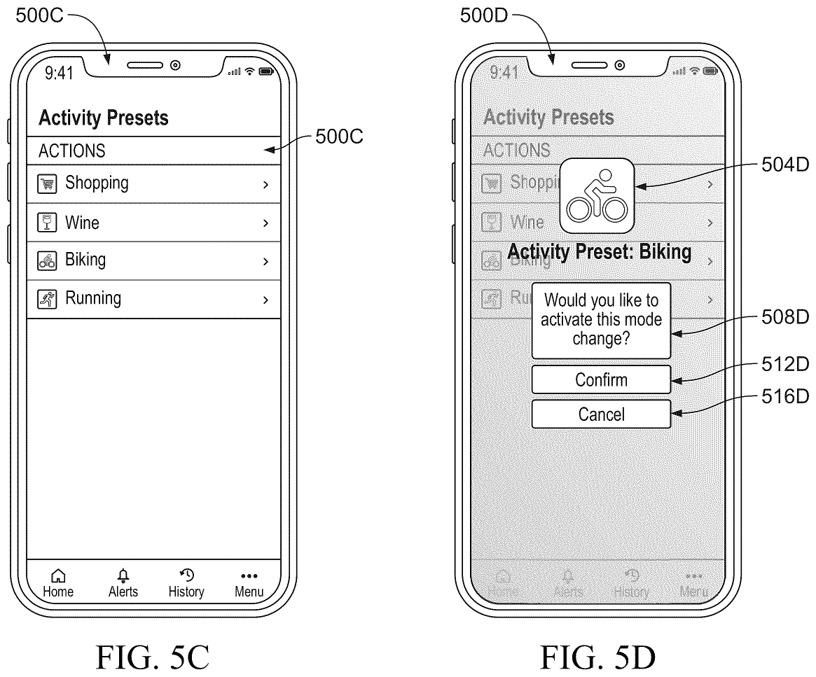

Referring now to FIG. 5A, a GUI 500A for activity mode selection is presented. GUI 500A may be similar to that of GUI 300A as described above with reference to FIG. 3A. GUI 500A may include glycemic data 500B, which may include glycemic data 404A as described above with reference to FIG. 3A. GUI 500A may include menu icon 508A. The menu icon 508A be displayed with three equally spaced dots in a row. The menu icon 508A may be positioned in a bottom corner of GUI 500A, such as a bottom right corner of GUI 500A. The menu icon 508A may include one or more text boxes. For instance, the menu icon 508A may include the word “More” displayed underneath a row of three dots.

Referring now to FIG. 5B, GUI 500B displaying a menu is illustrated. GUI 500B may include menu actions 504B. The menu actions 504B may include a list of 5 or more actions that may be selected through user input. For instance, and without limitation, the menu actions 504B may include a switch modes, medical device, blood glucose, pause medication, and/or activity presets action. The menu actions 504B may include a row arrangement, where each menu action is positioned in a rectangle on top of another menu action. The menu actions 504B may include a text box at a top of the menu actions 504B, such as a text box reading “Actions”.

Referring now to FIG. 5C, GUI 500C displaying activities menu 504C is presented. The activities menu 504C may include a list of two or more activity modes of a wearable medical device. For instance, the activities menu 504C may include a list of “shopping,” “wine,” “biking,” “running,” or other activity modes.

Referring now to FIG. 5D, GUI 500D displaying confirmation window 504D is presented. The confirmation window 504D may prompt a user with text, such as “Would you like to activate this mode change?”. The confirmation window 504D may be contrasted to a background of GUI 500D. For instance, a background of GUI 500D may have a lower brightness than that of the confirmation window 504D. The confirmation window 504D may include any windows as previously discussed. The confirmation window 504D may include activity icon 508D. The activity icon 508D may include a graphical representation of one or more activities. For instance, the activity icon 508D may include a pictorial representation of an individual riding a bike, which may be representative of a biking present mode. GUI 500D may include confirmation button 512D. Confirmation button 512D may include a rectangular window having a color, such as, but not limited to, orange. Confirmation button 512D may display one or more portions of text, such as letters, characters, numbers, symbols, and the like. In some embodiments, confirmation button 512D may display the word “Confirm”. A user may interact with the confirmation button 512D to confirm an activity mode. GUI 500D may display cancel icon 514D. The cancel icon 514D may include a rectangular icon that may display text, similar or the same to that of the confirmation button 512D. The cancel icon 514D may have a red color displaying white text that may read “Cancel”. A user may interact with the cancel icon 514D which may exit the confirmation window 504D. A user may interact with any of the above icons or other elements of GUI 500D through, but not limited to, touch input, mouse input, and the like.

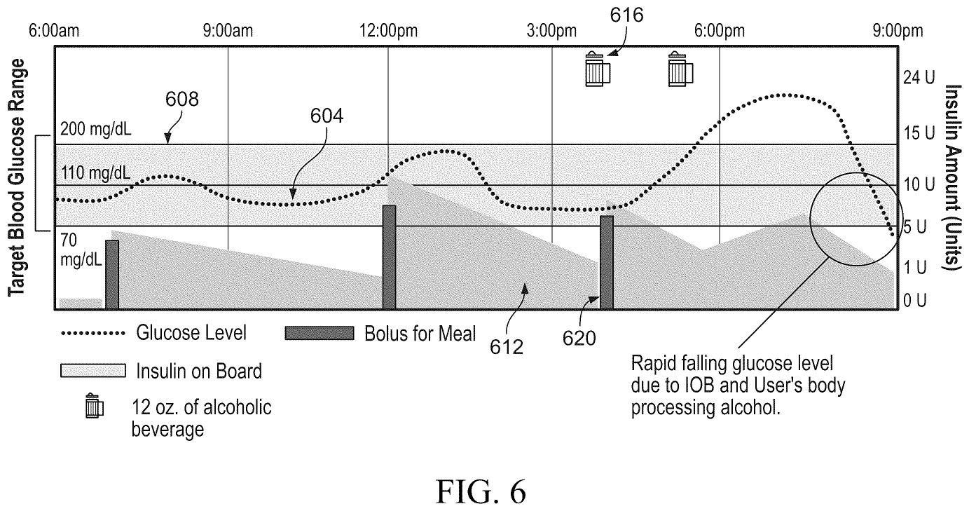

Referring now to FIG. 6, an exemplary graph 600 of blood glucose levels is shown. The graph 600 includes blood glucose level 604. Blood glucose level 604 may be a blood glucose concentration of an individual over a period of time. Graph 600 shows target blood glucose range 608. Target blood glucose range 608 may include a maximum and minimum blood glucose concentration of an individual that may be deemed safe. For instance, target blood glucose range 608 may include a maximum value of 200 mg/dL, a median value of 110 mg/d, and a low value of 70 mg/dL, without limitation. Graph 600 shows insulin on board levels 612. Insulin on board levels refer to the amount of insulin still in a user's body and not yet absorbed or that has yet to have an impact on the user's body. As shown in graph 600, over time, insulin on board levels 612 may decrease. In some embodiments, insulin on board levels 612 may decrease linearly. Graph 600 shows alcoholic beverage 616. The alcoholic beverage 616 may include, but is not limited to, 8 oz, 12 oz, 16 oz, and the like of an alcoholic drink. The alcoholic beverage 616 may include beer, for instance. Graph 600 shows bolus delivery 620. Bolus delivery 620 may include an amount of medication that may be delivered to a user, such as an amount of insulin.

Graph 600 shows an interaction between blood glucose levels 604, insulin on board levels 612, and alcoholic beverage 616. A user may consume one or more alcoholic beverages 616, which may increase blood glucose levels 604 rapidly. Blood glucose level 604 may drop rapidly after a user consumes alcoholic beverage 616. A drop in blood glucose level 604 may be attributed to a user's body processing alcohol and insulin on board levels 612 within a user's body. A user may enter into a hypoglycemic condition, such as below blood glucose target range 608 of 70 mg/dL.

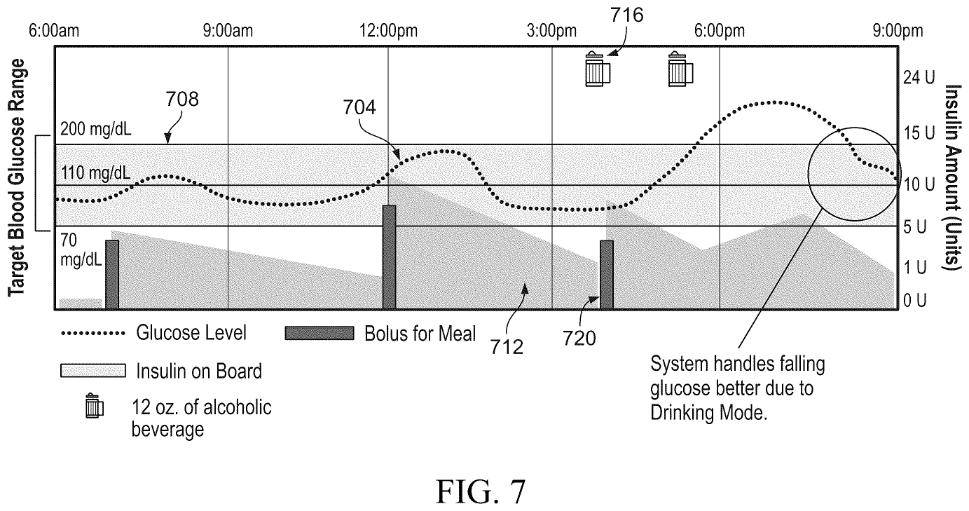

Referring now to FIG. 7, an exemplary graph illustrating blood glucose levels is shown. Graph 700 may include blood glucose levels 704, blood glucose range 708, insulin on board levels 712, alcoholic beverage 716, and/or bolus delivery 720, each of which may be the same as described above with reference to FIG. 6. Graph 700 shows that a user's blood glucose levels 704 may increase and decrease throughout a period of time. In some embodiments, a user's blood glucose levels 704 may rise and fall due to insulin on board levels 712, bolus delivery 724, and the like. The user may consume the alcoholic beverage 716, which may increase the user's blood glucose levels 704. A happy hour mode, such as the happy hour mode described above, may be activated, which may better handle a spike in the blood glucose levels 704 of the user. For instance, the user's blood glucose levels 704 may spike above 200 mg/dL of the blood glucose range 708 and then fall back into blood glucose range 708 at about 110 mg/dL. Insulin on board levels 712 may decrease as the user's blood glucose levels 704 increase.

FIG. 8 illustrates an exemplary embodiment of a drug delivery system Referring now to FIG. 8, a block diagram of a drug delivery system 800 is illustrated. In some examples, the drug delivery system 800 is suitable for delivering insulin to a user in accordance with the disclosed embodiments. The drug delivery system 800 may include a wearable injection device 802, a controller 804 and an analyte sensor 806. In addition, the drug delivery system may interact with a computing device 832 via a network 808 as well as interact with cloud-based services 810 via a wireless connection, such as a cellular data network or the like.

The wearable injection device 802 may be a wearable device that is worn on the body of the user. The wearable injection device 802 may be directly coupled to a user (e.g., directly attached to the skin of the user via an adhesive, or the like, at various locations on the user's body, such as thigh, abdomen, or upper arm). In an example, a surface of the wearable injection device 802 may include an adhesive to facilitate attachment to the skin of the user.

In an example, the wearable injection device 802 may include a processor 814. The processor 814 may be implemented in hardware, software, or any combination thereof. The processor 814 may, for example, be a microprocessor, a logic circuit, a field programmable gate array (FPGA), an application specific integrated circuit (ASIC) or a microprocessor coupled to a memory. The processor 814 may maintain a date and time as well as be operable to perform other functions (e.g., calculations or the like). The processor 814 may be operable to execute a control application 826 and a voice control application 888 stored in the memory 812 that enables the processor 814 to direct operation of the wearable drug delivery device 802. The control application 826 may control insulin delivery to the user utilizing an AID algorithm. The memory 812 may store settings 824 that may include AID application settings for a user, such as specific factor settings, subjective insulin need parameter settings, and AID algorithm settings, such as maximum insulin delivery, insulin sensitivity settings, total daily insulin (TDI) settings and the like. The memory 812 may also store other data 829, related to control and operation (e.g., status information of a power supply (not shown), reservoir level, event history, and operating history), and the like.

Still referring to FIG. 8, the input/output device(s) 845 may one or more of a microphone, a speaker, a vibration device, a display, a push button, a touchscreen display, a tactile input surface, or the like. The input/output device(s) 845 may be coupled to the processor 814 and may include circuitry operable to generate signals based on received inputs and provide the generated signals to the processor 814. In addition, the input/output device(s) 845 may be operable to receive signals from the processor 814 and, based on the received signals, generate outputs via a respective output device.

Still referring to FIG. 8, the wearable injection device 802 may include a reservoir 811. The reservoir 811 may be operable to store drugs, medications or therapeutic agents suitable for automated delivery, such as insulin, morphine, methadone, hormones, glucagon, glucagon-like peptide, blood pressure medicines, chemotherapy drugs, combinations of drugs, such as insulin and glucagon-like peptide, or the like. A fluid path to the user may be provided via tubing and a needle/cannula (not shown). The fluid path may, for example, include tubing coupling the wearable injection device 802 to the user (e.g., via tubing coupling a needle or cannula to the reservoir 811). The wearable injection device 802 may be operable based on control signals from the processor 814 to expel the drugs, medications or therapeutic agents, such as insulin, from the reservoir 811 to deliver doses of the drugs, medications or therapeutic agents, such as the insulin, to the user via the fluid path. For example, the processor 814 by sending control signals to the pump 818 may be operable to cause insulin to be expelled from the reservoir 811.

Still referring to FIG. 8, there may be one or more communication links 898 with one or more devices physically separated from the wearable drug delivery device 802 including, for example, a controller 804 of the user and/or a caregiver of the user and/or a sensor 806. The analyte sensor 806 may communicate with the wearable injection device 802 via a wireless communication link 831 and/or may communicate with the controller 804 via a wireless communication link 837. The communication links 831, 837, and 898 may include wired or wireless communication paths operating according to any known communications protocol or standard, such as Bluetooth, Wi-Fi, a near-field communication standard, a cellular standard, or any other wireless protocol.

Still referring to FIG. 8, the wearable injection device 802 may also include a user interface (UI) 816, such as an integrated display device for displaying information to the user, and in some embodiments, receiving information from the user. For example, the user interface 816 may include a touchscreen and/or one or more input devices, such as buttons, knob or a keyboard that enable a user to provide an input.

Still referring to FIG. 8, in addition, the processor 814 may be operable to receive data or information from the analyte sensor 806 as well as other devices, such as smart accessory device 830, fitness device 833 or another wearable device 834 (e.g., a blood oxygen sensor or the like), that may be operable to communicate with the wearable drug delivery device 802. For example, fitness device 833 may include a heart rate sensor and be operable to provide heart rate information or the like.

Still referring to FIG. 8, the wearable injection device 802 may interface with a network 808. The network 808 may include a local area network (LAN), a wide area network (WAN) or a combination therein and operable to be coupled wirelessly to the wearable injection device 802, the controller, and devices 830, 833, and 834. A computing device 832 may be interfaced with the network 808, and the computing device may communicate with the wearable injection device 802. The computing device 832 may be a healthcare provider device, a guardian's computing device, or the like through which a user's controller 804 may interact to obtain information, store settings, and the like. The AID application 820 may be operable to execute an AID algorithm and present a graphical user interface on the computing device 832 enabling the input and presentation of information related to the AID algorithm. The computing device 832 may be usable by a healthcare provider, a guardian of the user of the wearable injection device 802, or another user.

Still referring to FIG. 8, the drug delivery system 800 may include an analyte sensor 806 for detecting the levels of one or more analytes of a user, such as blood glucose levels, ketone levels, other analytes relevant to a diabetic treatment program, or the like. The analyte level values detected may be used as physiological condition data and be sent to the controller 804 and/or the wearable injection device 802. The sensor 806 may be coupled to the user by, for example, adhesive or the like and may provide information or data on one or more medical conditions and/or physical attributes of the user. The sensor 806 may be a continuous glucose monitor (CGM), ketone sensor, or another type of device or sensor that provides blood glucose measurements that is operable to provide blood glucose concentration measurements. The sensor 806 may be physically separate from the wearable injection device 802 or may be an integrated component thereof. The analyte sensor 806 may provide the processor 814 and/or processor 819 with physiological condition data indicative of measured or detected blood glucose levels of the user. The information or data provided by the sensor 806 may be used to modify an insulin delivery schedule and thereby cause the adjustment of drug delivery operations of the wearable injection device 802.

Still referring to FIG. 8, the analyte sensor 806 may be operable to collect physiological condition data, such as the blood glucose measurement values and a timestamp, ketone levels, heart rate, blood oxygen levels and the like that may be shared with the wearable injection device 802, the controller 804 or both. For example, the communication circuitry 842 of the wearable injection device 802 may be operable to communicate with the analyte sensor 806 and the controller 804 as well as the devices 830, 833 and 834. The communication circuitry 842 may be operable to communicate via Bluetooth®, Wi-Fi, a near-field communication standard, a cellular standard, or any other wireless protocol.

Still referring to FIG. 8, in the depicted example, the controller 804 may include a processor 819 and a memory 828. The controller 804 may be a special purpose device, such as a dedicated personal diabetes manager (PDM) device. The controller 804 may be a programmed general-purpose device that is a portable electronic device, such as any portable electronic device, smartphone, smartwatch, fitness device, tablet or the like including, for example, a dedicated processor, such as processor, a micro-processor or the like. The controller 804 may be used to program or adjust operation of the wearable injection device 802 and/or the sensor 806. The processor 819 may execute processes to manage a user's blood glucose levels and that control the delivery of the drug or a therapeutic agent (e.g., a liquid drug or the like as mentioned above) to the user. The processor 819 may also be operable to execute programming code stored in the memory 828. For example, the memory 828 may be operable to store an AID application 820 for execution by the processor 819. The AID application 820 may be responsible for controlling the wearable injection device 802, including the automatic delivery of insulin based on recommendations and instructions from the AID algorithm, such as those recommendations and instructions described herein.

Still referring to FIG. 8, the memory 828 may store one or more applications, such as an AID application 820, a voice control application, activity mode 888, and other data 839 which may be the same as, or substantially the same as those described above with reference to the wearable injection device 802. In addition, the settings 821 may store information, such as drug delivery history, blood glucose measurement values over a period of time, total daily insulin values, and the like. The memory 828 may be further operable to store other data 839, such as blood glucose history, medication delivery history, HbA1C history, programming code and libraries, and the like. In addition, the memory may store settings 821, which may include AID settings and parameters, insulin treatment program history (such as insulin delivery history, blood glucose measurement value history) and the like. Other parameters such as insulin-on-board (IOB) and insulin-to-carbohydrate ratio (ICR) may be retrieved from prior settings and insulin history stored in memory. For example, the control application 820 may be operable to store the AID algorithm settings, such as blood glucose target set points, insulin delivery constraints, basal delivery rate, insulin delivery history, wearable drug delivery device status, and the like. The memory 828 may also be operable to store data such as a food database for carbohydrate (or macronutrient) information of food components (e.g., grilled cheese sandwich, coffee, hamburger, brand name cereals, or the like). The memory 828 may be accessible to the AID application 820 and a voice control application.

Still referring to FIG. 8, the input/output device(s) 843 of the controller 804 may one or more of a microphone, a speaker, a vibration device, a display, a push button, a tactile input surface, or the like. The input/output device(s) 843 may be coupled to the processor 819 and may include circuitry operable to generate signals based on received inputs and provide the generated signals to the processor 819. In addition, the input/output device(s) 843 may be operable to receive signals from the processor 819 and, based on the received signals, generate outputs via one or more respective output devices, such as a speaker, a vibration device, or a display.

Still referring to FIG. 8, the controller 804 may include a user interface (UI) 823 for communicating visually with the user. The user interface 823 may include a display, such as a touchscreen, for displaying information provided by the AID application 820 or a voice control application. The touchscreen may also be used to receive input when it is a touch screen. The user interface 823 may also include input elements, such as a keyboard, button, knob or the like. In an operational example, the user interface 823 may include a touchscreen display controllable by the processor 819 and be operable to present the graphical user interface, and in response to a received input (audio or tactile), the touchscreen display is operable present a graphical user interface related to the received input.

Still referring to FIG. 8, the controller 804 may interface via a wireless communication link of the wireless communication links 898 with a network, such as a LAN or WAN or combination of such networks that provides one or more servers or cloud-based services 810 via communication circuitry 822. The communication circuitry 822, which may include transceivers 827 and 825, may be coupled to the processor 819. The communication circuitry 822 may be operable to transmit communication signals (e.g., command and control signals) to and receive communication signals (e.g., via transceivers 827 or 825) from the wearable injection device 802 and the analyte sensor 806. In an example, the communication circuitry 822 may include a first transceiver, such as 825, that may be a Bluetooth transceiver, which is operable to communicate with the communication circuitry 822 of the wearable injection device 802, and a second transceiver, such as 827, that may be a cellular transceiver, a Bluetooth® transceiver, a near-field communication transceiver, or a Wi-Fi transceiver operable to communicate via the network 808 with computing device 832 or with cloud-based services 810. While two transceivers 825 and 827 are shown, it is envisioned that the controller 804 may be equipped more or less transceivers, such as cellular transceiver, a Bluetooth transceiver, a near-field communication transceiver, or a Wi-Fi transceiver.

Still referring to FIG. 8, the cloud-based services 810 may be operable to store user history information, such as blood glucose measurement values over a set period of time (e.g., days, months, years), a drug delivery history that includes insulin delivery amounts (both basal and bolus dosages) and insulin delivery times, types of insulin delivered, indicated meal times, blood glucose measurement value trends or excursions or other user-related diabetes treatment information, specific factor settings including default settings, present settings and past settings, or the like.

Still referring to FIG. 8, other devices, like smart accessory device 830 (e.g., a smartwatch or the like), fitness device 833 and other wearable device 834 may be part of the drug delivery system 800. These devices may communicate with the wearable injection device 802 to receive information and/or issue commands to the wearable injection device 802. These devices 830, 833 and 834 may execute computer programming instructions to perform some of the control functions otherwise performed by processor 814 or processor 819. These devices 830, 833 and 834 may include user interfaces, such as touchscreen displays for displaying information such as current blood glucose level, insulin on board, insulin deliver history, or other parameters or treatment-related information and/or receiving inputs. The display may, for example, be operable to present a graphical user interface for providing input, such as request a change in basal insulin dosage or delivery of a bolus of insulin. Devices 830, 833 and 834 may also have wireless communication connections with the sensor 806 to directly receive blood glucose level data as well as other data, such as user history data maintained by the controller 804 and/or the wearable injection device 802.

Still referring to FIG. 8, the user interface 823 may be a touchscreen display controlled by the processor 819, and the user interface 823 is operable to present a graphical user interface that offers an input of a subjective insulin need parameter usable by the AID application 820. The processor 819 may cause a graphical user interface to be presented on the user interface 823. Different examples of the graphical user interface may be shown with respect to other examples. The AID application 820 may generate instructions for the pump 818 to deliver basal insulin to the user or the like.

Still referring to FIG. 8, the processor 819 is also operable to collect physiological condition data related to the user from sensors, such as the analyte sensor 806 or heart rate data, for example, from the fitness device 833 or the smart accessory device 830. In an example, the processor 819 executing the AID algorithm may determine a dosage of insulin to be delivered based on the collected physiological condition of the user and a specific factor determined based on the subjective insulin need parameter. The processor 819 may output a control signal via one of the transceivers 825 or 827 to the wearable drug delivery device 802. The outputted signal may cause the processor 814 to deliver command signals to the pump 818 to deliver an amount of related to the determined dosage of insulin in the reservoir 811 to the user based on an output of the AID algorithm. The processor 819 may also be operable to perform calculations regarding settings of the AID algorithm as discussed as herein. Modifications to the AID algorithm settings provided via the voice control application 821, such as by the examples described herein, may be stored in the memory 828.

Referring to FIG. 9, an exemplary machine-learning module 900 may perform machine-learning process(es) and may be configured to perform various determinations, calculations, processes and the like as described in this disclosure using a machine-learning process. The machine learning module 900 may utilize the training data 904. For instance, and without limitation, the training data 904 may include a plurality of data entries, each entry representing a set of data elements that were recorded, received, and/or generated together. The training data 904 may include data elements that may be correlated by shared existence in a given data entry, by proximity in a given data entry, or the like. Multiple data entries in the training data 904 may demonstrate one or more trends in correlations between categories of data elements. For instance, and without limitation, a higher value of a first data element belonging to a first category of data element may tend to correlate to a higher value of a second data element belonging to a second category of data element, indicating a possible proportional or other mathematical relationship linking values belonging to the two categories. Multiple categories of data elements may be related in the training data 904 according to various correlations. Correlations may indicate causative and/or predictive links between categories of data elements, which may be modeled as relationships such as mathematical relationships by machine-learning processes as described in further detail below. The training data 904 may be formatted and/or organized by categories of data elements. The training data 904 may, for instance, be organized by associating data elements with one or more descriptors corresponding to categories of data elements. As a non-limiting example, the training data 904 may include data entered in standardized forms by one or more individuals, such that entry of a given data element in a given field in a form may be mapped to one or more descriptors of categories. Elements in the training data 904 may be linked to descriptors of categories by tags, tokens, or other data elements. The training data 904 may be provided in fixed-length formats, formats linking positions of data to categories such as comma-separated value (CSV) formats and/or self-describing formats. Self-describing formats may include, without limitation, extensible markup language (XML), JavaScript Object Notation (JSON), or the like, which may enable processes or devices to detect categories of data.

With continued reference to refer to FIG. 9, the training data 904 may include one or more elements that are not categorized. Uncategorized data of the training data 904 may include data that may not be formatted or containing descriptors for some elements of data. In some embodiments, machine-learning algorithms and/or other processes may sort the training data 904 according to one or more categorizations. Machine-learning algorithms may sort the training data 904 using, for instance, natural language processing algorithms, tokenization, detection of correlated values in raw data and the like. In some embodiments, categories of the training data 904 may be generated using correlation and/or other processing algorithms. As a non-limiting example, in a body of text, phrases making up a number “n” of compound words, such as nouns modified by other nouns, may be identified according to a statistically significant prevalence of n-grams containing such words in a particular order. For instance, an n-gram may be categorized as an element of language such as a “word” to be tracked similarly to single words, which may generate a new category as a result of statistical analysis. In a data entry including some textual data, a person's name may be identified by reference to a list, dictionary, or other compendium of terms, permitting ad-hoc categorization by machine-learning algorithms, and/or automated association of data in the data entry with descriptors or into a given format. The ability to categorize data entries automatedly may enable the same the training data 904 to be made applicable for two or more distinct machine-learning algorithms as described in further detail below. The training data 904 used by the machine-learning module 900 may correlate any input data as described in this disclosure to any output data as described in this disclosure, without limitation.

Further referring to FIG. 9, the training data 904 may be filtered, sorted, and/or selected using one or more supervised and/or unsupervised machine-learning processes and/or models as described in further detail below. In some embodiments, the training data 904 may be classified using the training data classifier 916. The training data classifier 916 may include a classifier. A “classifier” as used in this disclosure is a machine learning model that sorts inputs into one or more categories. The training data classifier 916 may utilize a mathematical model, neural net, or program generated by a machine-learning algorithm. A machine learning algorithm of the training data classifier 916 may include a classification algorithm. A “classification algorithm” as used in this disclosure is one or more computer processes that generate a classifier from training data. A classification algorithm may sort inputs into categories and/or bins of data. A classification algorithm may output categories of data and/or labels associated with the data. A classifier may be configured to output a datum that labels or otherwise identifies a set of data that may be clustered together. The machine-learning module 900 may generate a classifier, such as the training data classifier 916 using a classification algorithm. Classification may be performed using, without limitation, linear classifiers such as without limitation logistic regression and/or naive Bayes classifiers, nearest neighbor classifiers such ask-nearest neighbors classifiers, support vector machines, least squares support vector machines, fisher's linear discriminant, quadratic classifiers, decision trees, boosted trees, random forest classifiers, learning vector quantization, and/or neural network-based classifiers. As a non-limiting example, the training data classifier 916 may classify elements of training data to one or activity modes.

Still referring to FIG. 9, the machine-learning module 900 may be configured to perform a lazy-learning process 920 which may include a “lazy loading” or “call-when-needed” process and/or protocol. A “lazy-learning process” may include a process in which machine learning is performed upon receipt of an input to be converted to an output, by combining the input and training set to derive the algorithm to be used to produce the output on demand. For instance, an initial set of simulations may be performed to cover an initial heuristic and/or “first guess” at an output and/or relationship. As a non-limiting example, an initial heuristic may include a ranking of associations between inputs and elements of the training data 904. Heuristic may include selecting some number of highest-ranking associations and/or the training data 904 elements. Lazy learning may implement any suitable lazy learning algorithm, including without limitation a K-nearest neighbors algorithm, a lazy naive Bayes algorithm, or the like. Persons skilled in the art, upon reviewing the entirety of this disclosure, will be aware of various lazy-learning algorithms that may be applied to generate outputs as described in this disclosure, including without limitation lazy learning applications of machine-learning algorithms as described in further detail below.

Still referring to FIG. 9, machine-learning processes as described in this disclosure may be used to generate the machine-learning models 924. A “machine-learning model” as used in this disclosure is a mathematical and/or algorithmic representation of a relationship between inputs and outputs, as generated using any machine-learning process including without limitation any process as described above, and stored in memory. For instance, an input may be sent to the machine-learning model 924, which once created, may generate an output as a function of a relationship that was derived. For instance, and without limitation, a linear regression model, generated using a linear regression algorithm, may compute a linear combination of input data using coefficients derived during machine-learning processes to calculate an output. As a further non-limiting example, the machine-learning model 924 may be generated by creating an artificial neural network, such as a convolutional neural network comprising an input layer of nodes, one or more intermediate layers, and an output layer of nodes. Connections between nodes may be created via the process of “training” the network, in which elements from a training data 904 set are applied to the input nodes, a suitable training algorithm (such as Levenberg-Marquardt, conjugate gradient, simulated annealing, or other algorithms) is then used to adjust the connections and weights between nodes in adjacent layers of the neural network to produce the desired values at the output nodes. This process is sometimes referred to as deep learning.

Still referring to FIG. 9, machine-learning algorithms may include the supervised machine-learning process 928. A “supervised machine learning process” as used in this disclosure is one or more algorithms that receive labelled input data and generate outputs according to the labelled input data. For instance, the supervised machine learning process 928 may include biological data as described above as inputs, activity modes as outputs, and a scoring function representing a desired form of relationship to be detected between inputs and outputs. A scoring function may maximize a probability that a given input and/or combination of elements inputs is associated with a given output to minimize a probability that a given input is not associated with a given output. A scoring function may be expressed as a risk function representing an “expected loss” of an algorithm relating inputs to outputs, where loss is computed as an error function representing a degree to which a prediction generated by the relation is incorrect when compared to a given input-output pair provided in the training data 904. Persons skilled in the art, upon reviewing the entirety of this disclosure, will be aware of various possible variations of the supervised machine-learning process 928 that may be used to determine relation between inputs and outputs. The supervised machine-learning processes may include classification algorithms as defined above.

Further referring to FIG. 9, machine learning processes may include the unsupervised machine-learning processes 932. An “unsupervised machine-learning process” as used in this disclosure is a process that calculates relationships in one or more datasets without labelled training data. The unsupervised machine-learning process 932 may be free to discover any structure, relationship, and/or correlation provided in the training data 904. The unsupervised machine-learning process 932 may not require a response variable. The unsupervised machine-learning process 932 may calculate patterns, inferences, correlations, and the like between two or more variables of the training data 904. In some embodiments, the unsupervised machine-learning process 932 may determine a degree of correlation between two or more elements of the training data 904.

Still referring to FIG. 9, the machine-learning module 900 may be designed and configured to create a machine-learning model 924 using techniques for development of linear regression models. Linear regression models may include ordinary least squares regression, which aims to minimize the square of the difference between predicted outcomes and actual outcomes according to an appropriate norm for measuring such a difference (e.g. a vector-space distance norm); coefficients of the resulting linear equation may be modified to improve minimization. Linear regression models may include ridge regression methods, where the function to be minimized includes the least-squares function plus term multiplying the square of each coefficient by a scalar amount to penalize large coefficients. Linear regression models may include least absolute shrinkage and selection operator (LASSO) models, in which ridge regression is combined with multiplying the least-squares term by a factor of I divided by double the number of samples. Linear regression models may include a multi-task lasso model wherein the norm applied in the least-squares term of the lasso model is the Frobenius norm amounting to the square root of the sum of squares of all terms. Linear regression models may include the elastic net model, a multi-task elastic net model, a least angle regression model, a LARS lasso model, an orthogonal matching pursuit model, a Bayesian regression model, a logistic regression model, a stochastic gradient descent model, a perceptron model, a passive aggressive algorithm, a robustness regression model, a Huber regression model, or any other suitable model that may occur to persons skilled in the art upon reviewing the entirety of this disclosure. Linear regression models may be generalized in an embodiment to polynomial regression models, whereby a polynomial equation (e.g. a quadratic, cubic or higher-order equation) providing a best predicted output/actual output fit is sought; similar methods to those described above may be applied to minimize error functions, as will be apparent to persons skilled in the art upon reviewing the entirety of this disclosure.

Continuing to refer to FIG. 9, machine-learning algorithms may include, without limitation, linear discriminant analysis. A machine-learning algorithm may include quadratic discriminate analysis. Machine-learning algorithms may include kernel ridge regression. Machine-learning algorithms may include support vector machines, including without limitation support vector classification-based regression processes. Machine-learning algorithms may include stochastic gradient descent algorithms, including classification and regression algorithms based on stochastic gradient descent. Machine-learning algorithms may include nearest neighbors algorithms. Machine-learning algorithms may include various forms of latent space regularization such as variational regularization. Machine-learning algorithms may include Gaussian processes such as Gaussian Process Regression. Machine-learning algorithms may include cross-decomposition algorithms, including partial least squares and/or canonical correlation analysis. Machine-learning algorithms may include naïve Bayes methods. Machine-learning algorithms may include algorithms based on decision trees, such as decision tree classification or regression algorithms. Machine-learning algorithms may include ensemble methods such as bagging meta-estimator, forest of randomized tress, AdaBoost, gradient tree boosting, and/or voting classifier methods. Machine-learning algorithms may include neural net algorithms, including convolutional neural net processes.

Software related implementations of the techniques described herein may include, but are not limited to, firmware, application specific software, or any other type of computer readable instructions that may be executed by one or more processors. Hardware related implementations of the techniques described herein may include, but are not limited to, integrated circuits (ICs), application specific ICs (ASICs), field programmable arrays (FPGAs), and/or programmable logic devices (PLDs). In some examples, the techniques described herein, and/or any system or constituent component described herein may be implemented with a processor executing computer readable instructions stored on one or more memory components.

In addition, or alternatively, while the examples may have been described with reference to a closed loop algorithmic implementation, variations of the disclosed examples may be implemented to enable open loop use. The open loop implementations allow for use of different modalities of delivery of insulin such as smart pen, syringe or the like. For example, the disclosed AP application and algorithms may be operable to perform various functions related to open loop operations, such as the generation of prompts requesting the input of information such as weight or age. Similarly, a dosage amount of insulin may be received by the AP application or algorithm from a user via a user interface. Other open-loop actions may also be implemented by adjusting user settings or the like in an AP application or algorithm.

Some examples of the disclosed device may be implemented, for example, using a storage medium, a computer-readable medium, or an article of manufacture which may store an instruction or a set of instructions that, if executed by a machine (i.e., processor or microcontroller), may cause the machine to perform a method and/or operation in accordance with examples of the disclosure. Such a machine may include, for example, any suitable processing platform, computing platform, computing device, processing device, computing system, processing system, computer, processor, or the like, and may be implemented using any suitable combination of hardware and/or software. The computer-readable medium or article may include, for example, any suitable type of memory unit, memory, memory article, memory medium, storage device, storage article, storage medium and/or storage unit, for example, memory (including non-transitory memory), removable or non-removable media, erasable or non-erasable media, writeable or re-writeable media, digital or analog media, hard disk, floppy disk, Compact Disk Read Only Memory (CD-ROM), Compact Disk Recordable (CD-R), Compact Disk Rewriteable (CD-RW), optical disk, magnetic media, magneto-optical media, removable memory cards or disks, various types of Digital Versatile Disk (DVD), a tape, a cassette, or the like. The instructions may include any suitable type of code, such as source code, compiled code, interpreted code, executable code, static code, dynamic code, encrypted code, programming code, and the like, implemented using any suitable high-level, low-level, object-oriented, visual, compiled and/or interpreted programming language. The non-transitory computer readable medium embodied programming code may cause a processor when executing the programming code to perform functions, such as those described herein.

The present disclosure furthermore relates to computer programs comprising instructions (also referred to as computer programming instructions) to perform the aforementioned functionalities. The instructions may be executed by a processor. The instructions may also be performed by a plurality of processors for example in a distributed computer system. The computer programs of the present disclosure may be for example preinstalled on, or downloaded to the medicament delivery device, management device, fluid delivery device, e.g. their storage.

Certain examples of the present disclosure were described above. It is, however, expressly noted that the present disclosure is not limited to those examples, but rather the intention is that additions and modifications to what was expressly described herein are also included within the scope of the disclosed examples. Moreover, it is to be understood that the features of the various examples described herein were not mutually exclusive and may exist in various combinations and permutations, even if such combinations or permutations were not made express herein, without departing from the spirit and scope of the disclosed examples. In fact, variations, modifications, and other implementations of what was described herein will occur to those of ordinary skill in the art without departing from the spirit and the scope of the disclosed examples. As such, the disclosed examples are not to be defined only by the preceding illustrative description.

Program aspects of the technology may be thought of as “products” or “articles of manufacture” typically in the form of executable code and/or associated data that is carried on or embodied in a type of non-transitory, machine readable medium. Storage type media include any or all of the tangible memory of the computers, processors or the like, or associated modules thereof, such as various semiconductor memories, tape drives, disk drives and the like, which may provide non-transitory storage at any time for the software programming. It is emphasized that the Abstract of the Disclosure is provided to allow a reader to quickly ascertain the nature of the technical disclosure. It is submitted with the understanding that it will not be used to interpret or limit the scope or meaning of the claims. In addition, in the foregoing Detailed Description, various features are grouped together in a single example for streamlining the disclosure. This method of disclosure is not to be interpreted as reflecting an intention that the claimed examples require more features than are expressly recited in each claim. Rather, as the following claims reflect, inventive subject matter lies in less than all features of a single disclosed example. Thus, the following claims are hereby incorporated into the Detailed Description, with each claim standing on its own as a separate example. In the appended claims, the terms “including” and “in which” are used as the plain-English equivalents of the respective terms “comprising” and “wherein,” respectively. Moreover, the terms “first,” “second,” “third,” and so forth, are used merely as labels and are not intended to impose numerical requirements on their objects.

The foregoing description of examples has been presented for the purposes of illustration and description. It is not intended to be exhaustive or to limit the present disclosure to the precise forms disclosed. Many modifications and variations are possible in light of this disclosure. It is intended that the scope of the present disclosure be limited not by this detailed description, but rather by the claims appended hereto. Future filed applications claiming priority to this application may claim the disclosed subject matter in a different manner and may generally include any set of one or more limitations as variously disclosed or otherwise demonstrated herein.

What is claimed is:

1. An apparatus for glycemic control of a user, comprising:
a display device;
a processor in electronic communication with the display device; and
a memory communicatively connected to the processor, the memory containing instructions configuring the processor to:
generate a user interface through the display device, wherein the user interface is configured to receive user input;
receive biological data of the user from a biological sensor in communication with the processor; and
select an activity mode of a plurality of activity modes of a wearable injection device based on at least one of the user input and the biological data, the activity mode indicative of a temporary condition affecting a blood glucose level of the user;
modify a set of operational parameters for calculating medication dosages based on the selected activity mode;
calculate an amount of medication to deliver to the user based on the biological data and the selected activity mode.

2. The apparatus of claim 1, wherein the processor is further configured to:
correlate the biological data with an activity mode of the plurality of activity modes; and
prompt the user, through the user interface, to select the correlated activity mode.

3. The apparatus of claim 1, wherein the processor is further configured to:
train an activity mode machine learning model with the training data, wherein the activity mode machine learning model is configured to input biological data and output an activity mode; and
determine an activity mode based on the output of the activity mode machine learning model.

4. The apparatus of claim 1, wherein the processor is further configured to:
receive data from an external computing device of a plurality of biological data associated with a plurality of activity modes;
calculate an average selected activity mode based on the plurality of biological data; and
select an activity mode for the user based on the average selected activity mode.

5. The apparatus of claim 1, wherein the processor is further configured to:
receive an acuteness factor through the user interface; and
modify the activity mode based on the acuteness factor.

6. The apparatus of claim 1, wherein the processor is further configured to:
determine a pattern of the user based the biological data; and
present an activity mode to the user through the user interface based on the pattern.

7. The apparatus of claim 1, wherein the processor is further configured to:
generate a list of activity modes; and
present the list of activity modes to the user through the user interface.

8. The apparatus of claim 1, wherein the amount of medication includes insulin.

9. The apparatus of claim 1, wherein the biological sensor includes a blood glucose meter and a blood alcohol sensor.

10. A system for glycemic control, comprising:
a biological sensor configured to generate biological data;
a wearable medical device comprising:
a liquid reservoir, wherein the liquid reservoir stores medication; and
an injector, wherein the injector is configured to administer the medication from the liquid reservoir to a user; and
a computing device in electronic communication with the wearable medical device, wherein the computing device is configured to:
receive the biological data from the biological sensor;
generate a user interface through a display device in electronic communication with the computing device;
receive user input from the user interface;
select an activity mode of a plurality of activity modes of the wearable medical device based on at least one of the biological data and the user input, the activity mode indicative of a temporary condition affecting blood glucose levels of the user;
modify a set of operational parameters for calculating medication dosages based on the selected activity mode; and
communicate the selected activity mode with the wearable medical device, wherein the wearable medical device administers the medication to the user based on the selected activity mode.

11. The system of claim 10, wherein the computing device is further configured to:
correlate the biological data with an activity mode of the plurality of activity modes; and
prompt the user, through the user interface, to select the correlated activity mode.

12. The system of claim 10, wherein the computing device is further configured to:
train an activity mode machine learning model with the training data, wherein the activity mode machine learning model is configured to input biological data and output an activity mode; and
determine an activity mode based on the output of the activity mode machine learning model.

13. The system of claim 10, wherein the computing device is further configured to:
receive data from an external computing device of a plurality of biological data associated with a plurality of activity modes;
calculate an average selected activity mode based on the plurality of biological data; and
select an activity mode for the user based on the average selected activity mode.

14. The system of claim 10, wherein the computing device is further configured to:
receive an acuteness factor through the user interface; and
modify the activity mode based on the acuteness factor.

15. The system of claim 10, wherein the computing device is further configured to:
determine an activity pattern of the user based on the selected activity mode and biological data; and
present an activity mode to the user through the user interface based on the activity pattern.

16. The system of claim 10, wherein the computing device is further configured to:
generate a list of activity modes; and
present the list of activity modes to the user through the user interface.

17. The system of claim 10, wherein the activity mode includes a drinking mode, which presumes the user consuming one or more alcoholic beverages.

18. The system of claim 17, wherein the drinking mode includes a hardness factor of the one or more alcoholic beverages.

19. The system of claim 10, wherein the medication includes insulin.

20. The system of claim 10, wherein the biological sensor includes a blood glucose meter and a blood alcohol sensor.
